(19) United States
(12) Patent Application Publication
(10) Pub. No.: US 2008/0294008 A1
(43) Pub. Date: Nov. 27, 2008

(54) ENDOSCOPE

(75) Inventor: Ryuichi TOYAMA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/119,808

(22) Filed: May 13, 2008

(30) Foreign Application Priority Data
May 22, 2007 (JP) 2007-135865

(51) Int. Cl. A61B 1/012; A61B 1/005; A61B 1/06

(52) U.S. Cl. 600/139; 600/156; 600/158

(57) ABSTRACT

An endoscope according to the present invention includes an insertion portion that is inserted into a duct, an observation window provided at a distal end in the insertion direction of the insertion portion for observing the inside of the duct, a balloon provided so as to extend in a radial shape in a diametrical direction of the insertion portion with respect to the insertion portion and which contacts an inner wall of the duct to fix the insertion portion to the inner wall, and a plurality of suction holes that are provided at a position that is further on the distal end side in the insertion direction than the balloon while being closer to a proximal end side in the insertion direction than the observation window in the insertion portion and that suck in dirt removed from the inner wall by contact between the balloon and the inner wall.

This application claims benefit of Japanese Patent Application No. 2007-135865 filed in Japan on May 22, 2007, the contents of which are incorporated by this reference.

### BACKGROUND OF THE INVENTION

1. Field of the Invention

The present invention relates to an endoscope that is equipped with an insertion portion that is inserted into a duct, and which is provided with an observation window that enables observation within the duct at a distal end in the insertion direction of the insertion portion.

2. Description of the Related Art

In recent years, endoscopes have been widely used in medical treatment fields and industrial fields. Endoscopes enable observation inside a duct by insertion of a long and narrow insertion portion into the duct.

Further, for example, as necessary an endoscope used in the field of medical treatment makes it possible to perform various kinds of treatment on an examination site inside a body cavity by using a treatment instrument that is inserted into a channel provided in the endoscope.

Generally, an observation window that is used for observing inside a duct or an illumination window that emits an illumination light to the inside of the duct are provided in the distal end surface of a rigid distal end portion that is provided at the distal end side of the insertion portion of the endoscope. Hence, when inserting an insertion portion into a duct to perform endoscopic observation, the inside of the duct that is illuminated by illumination light from the illumination window can be observed from the observation window.

In this case, when performing observation of an examination site inside a duct using an observation window, or when performing various treatments at an examination site inside a duct, the insertion portion is inserted through the duct to a position at which the observation window of the distal end portion faces the examination site. However, there is a problem that if the distal end portion moves after inserting the insertion portion to the examination site, it is difficult to observe the examination site and perform various kinds of treatment.

In view of this kind of problem, a configuration is known that enables observation or various kinds of treatment of an examination site to be stably performed by fixing a distal end portion of an endoscope to an inner wall of a duct through a balloon. This is achieved by providing a balloon that is a fixing member that can freely expand or contract along with feeding or discharging a fluid, at the outer periphery of the distal end portion and, at the time of observation, inflating the balloon to cause the balloon to contact the inner wall of the duct with a pressure.

Further, when advancing the insertion portion inside a flexible duct, in particular when advancing the insertion portion inside a body cavity, for example, inside the large intestine, if the distal end portion is collapsed by an intestinal wall such as at a site where the large intestine bends, not only is it difficult to perform insertion, but it is also difficult for the technician to figure out in which direction the distal end portion is advancing.

However, at this time also, by advancing the insertion portion in a state in which the aforementioned balloon is inflated, the outer periphery of the distal end portion does not contact the intestinal wall and thus collapse of the distal end portion by the intestinal wall can be prevented. In this connection, the above situation is similar to a case in which insertion of the insertion portion is performed by a technician and a case in which insertion thereof is performed using an automatic insertion device.

However, when the insertion portion is advanced while a balloon is inflated, the balloon comes in contact with an inner wall within the duct. As a result, dirt that adheres to the inner wall within the duct is removed on the distal end side in the insertion direction by the balloon. As a result, dirt accumulates on the distal end surface side of the distal end portion and adheres to the observation window and the illumination window. There is, thus, the problem that performing observation and illuminating inside the duct become difficult.

In consideration of this problem, Japanese Patent Laid-Open No. 1-249041 and Japanese Patent Laid-Open No. 2005-270216 disclose configurations in which a balloon is provided at the furthermost distal end side in the insertion direction on the outer periphery of the distal end portion so that the balloon is thus located adjacent to an opening of a suction duct which is passed through the insertion portion that opens at the distal end surface of the distal end portion.

In this connection, for example in the case of an endoscope for medical treatment use, since the purpose of the suction duct opening is to suck in body fluids in the periphery of the examination site and excised tissue of an examination site, the suction duct opening is generally provided at the distal end surface.

By utilizing this configuration, the dirt that is removed due to contact between the balloon and the inner wall of the duct can be sucked in from the suction duct opening, and it is thus possible to prevent the dirt from adhering to the observation window and the illumination window.

### SUMMARY OF THE INVENTION

Briefly, an endoscope according to the present invention comprises an insertion portion that is inserted into a duct; an observation window that is provided at the distal end in the insertion direction of the insertion portion for observing inside the duct; a fixing member that is provided so as to expand in a radial shape in the diametrical direction of the insertion portion with respect to the insertion portion and which contacts an inner wall of the duct and fixes the insertion portion to the inner wall; and a suction portion provided in the insertion portion at a position further on the distal end side in the insertion direction than the fixing member and further on the proximal end side in the insertion direction than the observation window and which sucks in dirt that is removed from the inner wall by contact of the fixing member.

Further, an endoscope according to the present invention comprises an insertion portion that is inserted into a duct; an illumination light emitting window that is provided at the distal end in the insertion direction of the insertion portion for illuminating the inside of the duct; a fixing member provided so as to expand in a radial shape in the diametrical direction of the insertion portion with respect to the insertion portion and which contacts an inner wall of the duct and fixes the insertion portion to the inner wall; and a suction portion provided in the insertion portion at a position further on the distal end side in the insertion direction than the fixing member and further on the proximal end side in the insertion direction than the illumination light emitting window and which sucks in dirt that is removed from the inner wall by contact of the fixing member.

The above and other objects, feature and advantages of the invention will become more clearly understood from the following description referring to the accompanying drawings.

### BRIEF DESCRIPTION OF THE DRAWINGS

FIG. 1 is a diagram showing an endoscope apparatus comprising an endoscope according to the present embodiments;

FIG. 2 is a diagram showing an expanded view of the distal end side in the insertion direction of an insertion portion of the endoscope shown in FIG. 1;

FIG. 3 is a cross-sectional view taken along a line III-III in FIG. 2;

FIG. 4 is a cross-sectional view taken along a line IV-IV in FIG. 2;

FIG. 5 is a diagram showing a modification example in which a balloon and a plurality of suction holes are provided on the proximal end side of a bending portion of the insertion portion of the endoscope, as well as the distal end side in the insertion direction of the insertion portion;

FIG. 6 is a diagram showing a modification example in which a plurality of suction holes shown in FIG. 2 are formed as long elliptical holes in the circumferential direction of the distal end portion;

FIG. 7 is a diagram showing a modification example in which the endoscope according to the present embodiments is applied to a double-balloon endoscope;

FIG. 8 is a diagram showing a modification example in which the endoscope according to the present embodiments is applied to a self-propelled double-balloon endoscope;

FIG. 9 is a perspective view that illustrates an opening portion of an air supply conduit that is formed in a distal end surface of an oversheath shown in FIG. 8; and

FIG. 10 is a diagram showing a modification example in which an endoscope according to the present embodiments is applied to a self-propelled endoscope.

### DETAILED DESCRIPTION OF THE PREFERRED EMBODIMENTS

Hereunder, an embodiment of the present invention is described with reference to the drawings. In the present embodiment, an endoscope is described by taking the example of an endoscope for medical treatment use. Hence, a case is described in which a duct into which the endoscope is inserted is taken as a body cavity.

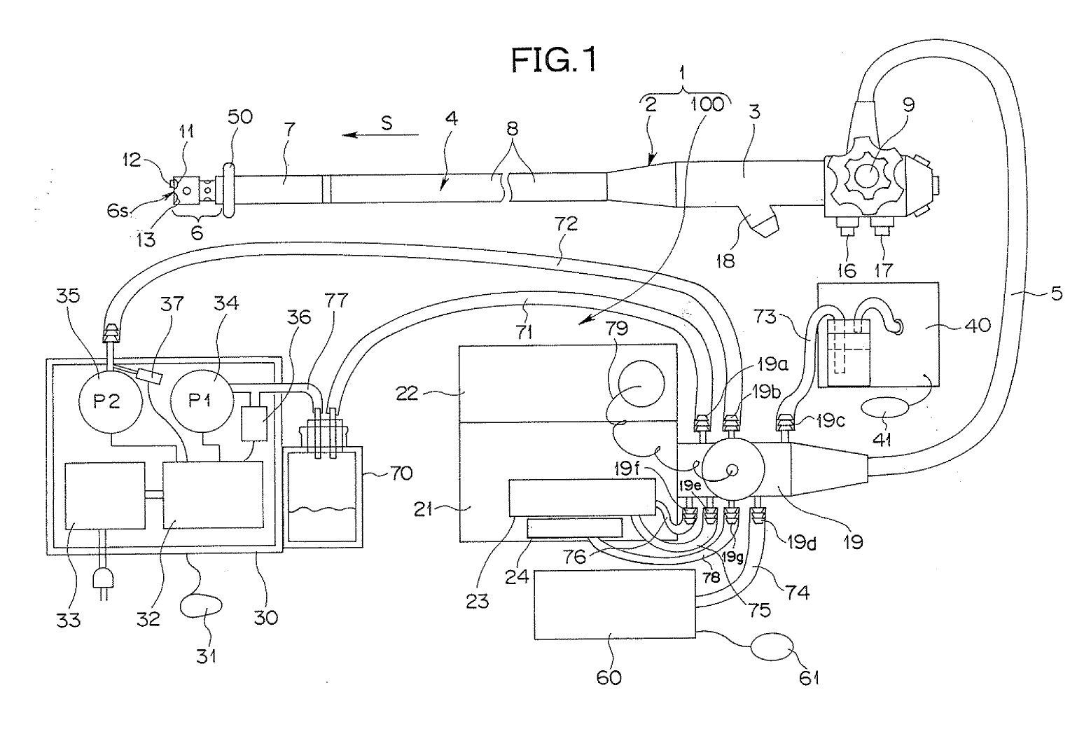

FIG. 1 is a diagram that shows an endoscope apparatus comprising an endoscope according to the present embodiment.

As shown in FIG. 1, an endoscope apparatus 1 comprises an endoscope 2 and a peripheral device 100. The endoscope 2 comprises an operation portion 3, an insertion portion 4, a universal cord 5, and an endoscope connector 19 as principal components.

The peripheral device 100 comprises an electric light source 21, a video processor 22, a suction pump unit 30 that is a suction apparatus, a water supply pump unit 40, and a balloon controller 60 as principal components. The peripheral device 100 and the endoscope 2 are connected to each other at an endoscope connector 19 by various tubes and the like that are described later.

In the operation portion 3 of the endoscope 2 are provided a bending operation knob 9, an air supply/water supply operation button 16, a suction operation button 17, and a treatment instrument insertion opening 18.

The insertion portion 4 of the endoscope 2 comprises, in order from the distal end side in the insertion direction S, a distal end portion 6 that is a distal end rigid portion, a bending portion 7, and a flexible tubular portion 8. The bending portion 7 is a portion that causes the distal end portion 6 to be oriented in a plurality of directions, for example, upward and downward or right and left, by applying a bending operation by the bending operation knob 9 that is provided in the operation portion 3.

On a distal end surface 6s on the distal end side in the insertion direction S of the distal end portion 6 is provided an observation window 11 that is an objective lens which is located furthest on the distal end side in the insertion direction S in a group of objective lenses (none of these are shown in the figure) configuring an image pickup unit that is disposed inside the distal end portion 6.

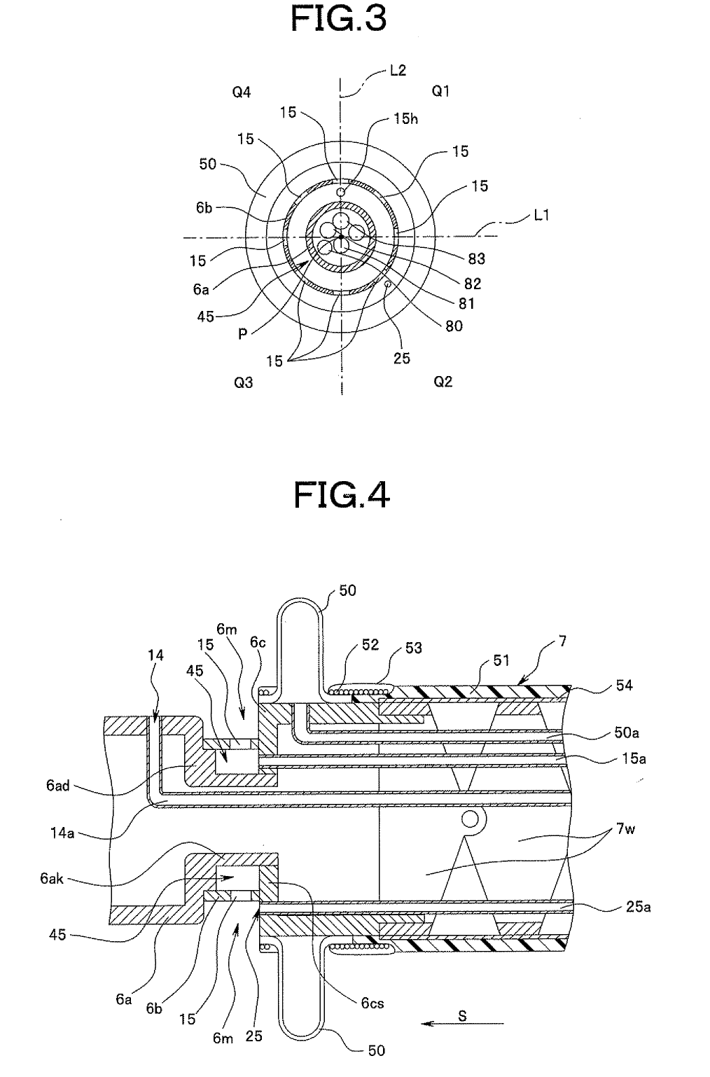

Further, on the distal end surface 6s of the distal end portion 6 are disposed a nozzle 12 that cleans the surface of the observation window 11 that is provided on the distal end surface 6s by blowing out a fluid such as water and air at the surface of the observation window 11, an illumination light emitting window (hereunder, referred to simply as “illumination window”) 13 that irradiates an illumination light into the body cavity, and a second suction portion (not shown) that forms an opening of the distal end side in the insertion direction S in a second suction duct 83 described later (see FIG. 3) and also serves as a treatment instrument insertion passage (not shown) that communicates with the treatment instrument insertion opening 18.

When a button operation of the air supply/water supply operation button 16 of the operation portion 3 is performed, a gas and a liquid are selectively blown out from the nozzle 12. When a button operation of the suction operation button 17 of the operation portion 3 is performed, mucous that is inside the body cavity or liquid or the like that is ejected from the nozzle 12 is selectively collected from the second suction portion.

In the endoscope connector 19 that is disposed at the distal end of the universal cord 5 of the endoscope 2 is provided an electric contact portion or the like to which a plurality of mouthpieces 19a to 19g, an unshown light guide mouthpiece configuring the end of a light guide 81 that is described later (see FIG. 3), and an end of an image pickup cable 80 that is described later (see FIG. 3) are connected.

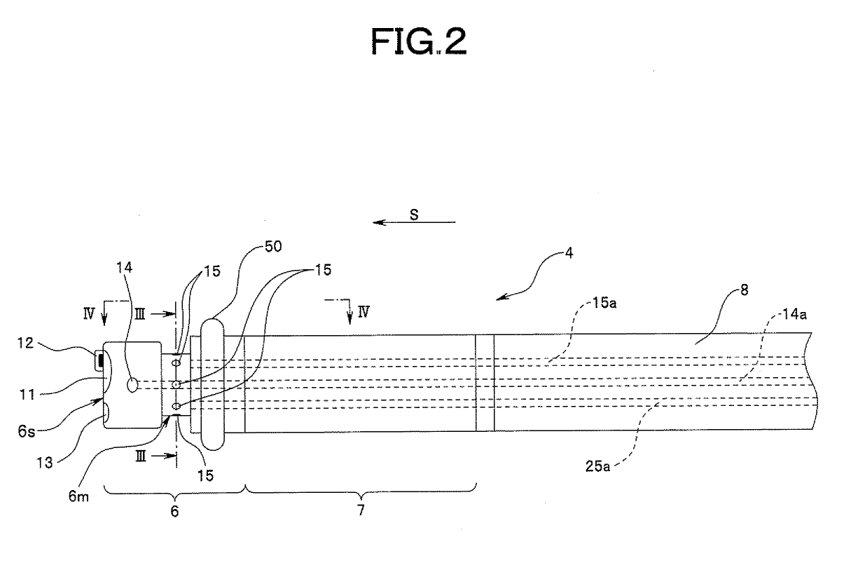

The mouthpiece 19a is connected to an end of the endoscope connector 19 side of a suction duct 15a that is described later (see FIG. 2) and is also connected to a suction bottle 70 via a suction tube 71.

The suction bottle 70 is connected to a suction pump (P1) 34 that is a suction pressure changing portion of the suction pump unit 30 through a tube 77. At a midway position along the tube 77 inside the suction pump unit 30 is provided a pressure sensor 36 as a suction amount detection portion and a detection portion.

The mouthpiece 19b connects to an end of the endoscope connector 19 side of an air supply conduit 14a that is described later (see FIG. 2) and also connects to the air supply pump (P2) 35 of the suction pump unit 30 through an air supply tube 72. At a midway position between the air supply tube 72 and an air supply pump 35 inside the suction pump unit 30 is provided a pressure sensor 37 as an air supply amount detection portion.

Inside the suction pump unit 30, a suction pump 34, the air supply pump 35, the pressure sensor 36, and the pressure sensor 37 are electrically connected to a control circuit 32 that is an air supply amount control portion and a suction pressure control circuit. The control circuit 32 is electrically connected to a power circuit 33. Further, the suction pump unit 30 is electrically connected to a foot switch 31.

When the suction pump unit 30 is turned on by the power circuit 33 and an operation signal from the foot switch 31 is input thereto, the suction pump unit 30 sucks in dirt of the inner wall inside the body cavity by driving control of the suction pump 34 by the control circuit 32 from a plurality of suction holes 15 (see FIG. 2) that are described later that open at the distal end portion 6 through the tube 77, the suction tube 71, the mouthpiece 19a, and the suction duct 15a (see FIG. 2) that is passed through the inside of the insertion portion 4, the operation portion 3, the universal cord 5, and the endoscope connector 19.

At this time, the control circuit 32 controls the suction pressure of the suction pump 34 by detecting the suction pressure of the suction pump 34 using the pressure sensor 36. Further, when the pressure sensor 36 detects that the suction pressure is too high, the control circuit 32 executes control to stop the suction pump 34.

Furthermore, by performing detection of the suction pressure using the pressure sensor 36, it is possible to prevent a situation from occurring in which the suction pump 34 executes suction from the plurality of suction holes 15 at a higher pressure than normal, thereby causing the plurality of suction holes 15 to suck in the inner wall in the body cavity. More specifically, the suction pump 34 can perform suction at the appropriate pressure.

Dirt that is sucked in is accumulated in the suction bottle 70. Suction using the suction pump 34 does not depend on input of an operation signal from the foot switch 31, and while the endoscope 2 is inserted into the body cavity, for example, may be automatically performed while an image pickup device that is provided in the distal end portion 6 of the endoscope 2 is driving.

When a suction operation by the suction pump 34 is started, the suction pump unit 30 automatically feeds a gas into the body cavity from an air supply opening portion 14 (see FIG. 2), described later, that opens at the distal end portion 6 through the air supply tube 72, the mouthpiece 19b, and the air supply conduit 14a (see FIG. 2) that is passed through the inside of the insertion portion 4, the operation portion 3, the universal cord 5, and the endoscope connector 19, by driving control of the air supply pump 35 by the control circuit 32.

Feeding of a gas using the air supply pump 35 by the control circuit 32 is configured to be automatically performed with respect to the amount of the gas that is sucked in with the suction pump 34 by detecting the air supply pressure using the pressure sensor 37. That is, gas is fed from the air supply pump 35 at the same pressure as the suction pressure of the suction pump 34.

Further, feeding of gas into the body cavity by the air supply pump 35 is performed to prevent the lumen inside the body cavity from contracting along with the suction of dirt within the body cavity by the suction pump 34. As a result, when performing suction, not only can optimal swelling of the lumen always be ensured to enhance the insertability of the insertion portion 4, but the observational field of view from the observation window 11 can also be ensured. Further, since supply of air is automatically performed, and it is not necessary for the operator to perform an air supply operation every time that dirt is sucked in, the detection efficiency is improved.

The mouthpiece 19c connects to an end of the endoscope connector 19 side of a water supply conduit 25a that is described later (see FIG. 2) and also connects to the water supply pump unit 40 through a tube 73.

The water supply pump unit 40 is electrically connected to a foot switch 41. In accordance with an input operation from the foot switch 41, the water supply pump unit 40 supplies a liquid to high-viscosity dirt that accumulates in a groove portion 6m (see FIG. 2) that is described later of the distal end portion 6 from a water supply conduit opening portion 25, described later, that opens at the distal end portion 6 via the tube 73, the mouthpiece 19c, and the water supply conduit 25a (see FIG. 2) that is passed through the inside of the insertion portion 4, the operation portion 3, the universal cord 5 and the endoscope connector 19, to thereby soften dirt that is difficult to be sucked in from the plurality of suction holes 15.

The mouthpiece 19d connects to an end of the endoscope connector 19 side of a balloon duct 50a that is described later (see FIG. 4) and also connects to the balloon controller 60 through a tube 74.

The balloon controller 60 is electrically connected to a foot switch 61. In accordance with an input operation from the foot switch 61, the balloon controller 60 expands or contracts a balloon 50, described later, that is provided at the distal end portion 6 by supplying a gas to the balloon 50 via the tube 74, the mouthpiece 19d, and the balloon duct 50a (see FIG. 4) that is passed through the inside of the insertion portion 4, the operation portion 3, the universal cord 5 and the endoscope connector 19 or by evacuating a gas from the balloon 50 using a pressure release valve.

The mouthpiece 19e and mouthpiece 19f connect to an end of the endoscope connector 19 side of an air supply/water supply conduit 82 that is described later (see FIG. 3) and also connect to an air supply/water supply pump 23 that is provided inside the electric light source 21 through a tube 75 for liquid feeding and a tube 76 for gas feeding, respectively.

In accordance with an input operation from the air supply/water supply operation button 16 of the operation portion 3, the air supply/water supply pump 23 selectively supplies a gas or a liquid to the observation window 11 from the nozzle 12 via the tube 75 or tube 76, the mouthpiece 19e or mouthpiece 19f, and the air supply/water supply conduit 82 that is passed through the inside of the insertion portion 4, the operation portion 3, the universal cord 5, and the endoscope connector 19.

The mouthpiece 19g connects to an end of the endoscope connector 19 side of the second suction duct 83 and also connects to a suction pump 24 that is provided inside the electric light source 21 through a tube 78.

In accordance with an input operation from the suction operation button 17 of the operation portion 3, the suction pump 24 selectively collects mucus or the like that is inside the body cavity from a second suction portion that opens at the distal end surface 6s of the distal end portion 6 via the tube 78, the mouthpiece 19g, and the second suction duct 83 that is passed through the inside of the insertion portion 4, the operation portion 3, the universal cord 5, and the endoscope connector 19.

The aforementioned light guide mouthpiece of the endoscope connector 19 is connected to the electric light source 21. The light guide 81 (see FIG. 3) is passed from the light guide mouthpiece through the inside of the universal cord 5, the inside of the operation portion 3, and the inside of the insertion portion 4 to a position adjacent to the illumination window 13 inside the distal end portion 6. The light guide 81 sends an illumination light from the electric light source 21 to the illumination window 13, and expands and radiates the illumination light into the body cavity through the illumination window 13.

The aforementioned electric contact portion of the endoscope connector 19 is electrically connected to a video processor 22 by a cable 79. An image pickup cable 80 (see FIG. 3) is passed from the electric contact portion through the inside of the universal cord 5, the inside of the operation portion 3, and the inside of the insertion portion 4 to an unshown image pickup apparatus that is provided inside the distal end portion 6.

The image pickup cable 80 transmits electrical signals of images of an examination site within the body cavity that are picked up through the observation window 11 with the image pickup apparatus to the video processor 22, and also transmits instruction signals from the video processor 22 to the image pickup apparatus.

The video processor 22 performs predetermined image processing on the electrical signals that are transmitted by the image pickup cable 80 to thereby display images of inside the body cavity on an unshown monitor, and also performs various kinds of image pickup control on the image pickup apparatus via the image pickup cable 80.

Next, the configuration of the distal end side in the insertion direction S of the insertion portion 4 of the endoscope 2 is described using FIG. 2 to FIG. 4. FIG. 2 is a diagram showing an expanded view of the distal end side in the insertion direction of the insertion portion of the endoscope shown in FIG. 1. FIG. 3 is a cross sectional view taken along a line III-III in FIG. 2. FIG. 4 is a cross sectional view taken along a line IV-IV in FIG. 2.

As shown in FIG. 4, in the distal end portion 6, the principal portions are configured by a rigid first tubular portion 6a, a second tubular portion 6b that has a smaller diameter than the first tubular portion 6a, and a third tubular portion 6c that has a larger diameter than the first tubular portion 6a and the second tubular portion 6b, that are respectively annular.

The distal end in the insertion direction S of the second tubular portion 6b is hermetically fitted to the outer periphery of a stepped portion 6ad of the proximal end side in the insertion direction S of the first tubular portion 6a. Further, a distal end surface 6cs having a flange shape inwardly extending at the distal end in the insertion direction S in the third tubular portion 6c is hermetically fitted to the outer periphery of a mouthpiece 6ak on a proximal end side in the insertion direction S of the first tubular portion 6a. Furthermore, the proximal end in the insertion direction S of the second tubular portion 6b is hermetically connected to the distal end surface 6cs.

Of a plurality of bending dies 7w of a bending portion 7, a bending die 7w at the distal end in the insertion direction S is fixed to a mouthpiece at a proximal end portion in the insertion direction S of the third tubular portion 6c. The outer periphery of the plurality of bending dies 7W is covered by a mesh pipe 54. Further, the outer periphery of the proximal end side in the insertion direction S of the third tubular portion 6c and the outer periphery of the mesh pipe 54 of the bending portion 7 are covered by a bending rubber tube 51.

Furthermore, as shown in FIG. 4, a balloon 50 as a fixing member is provided at the outer periphery of the third tubular portion 6c at the distal end portion 6 of the insertion portion 4 such that the balloon 50 extends in a radial shape in the diametrical direction of the insertion portion 4. Along with the distal end side in the insertion direction S of the bending rubber tube 51, the balloon 50 is also fixed to the outer periphery of the third tubular portion 6c by winding with a thread 52 and an adhesive 53.

The balloon 50 is a member that is expandable and contractible in the diametrical direction in accordance with feeding or releasing a gas through the tube 74, the mouthpiece 19d, and the balloon duct 50a by the balloon controller 60. At a time of expansion, the balloon 50 contacts the inner wall within the body cavity to fix the insertion portion 4 to the inner wall and facilitates advance of the insertion portion 4 to a deep part inside the body cavity.

Because the balloon 50 is formed by a flexible member, when the balloon 50 is expanded it is possible to decrease a change in shape within the body cavity, for example, the intestines, thereby reducing the pain of the examinee.

Further, since the balloon 50 is expandable and contractible in the diametrical direction, it is possible to adjust the balloon diameter in accordance with the state inside the body cavity, for example, within the intestines. At the position of the third tubular portion 6c at which the balloon 50 is fixed, a communicating hole for causing the inside of the balloon 50 and the inside of the balloon duct 50a to communicate is formed in the diametrical direction.

Further, at the outer peripheral surface along the insertion direction S of the distal end portion 6, at positions that are further on the distal end side in the insertion direction S than the balloon 50 and further on the proximal end side in the insertion direction S than the observation window 11 or the illumination window 13 provided on the distal end surface 6s are provided a plurality of suction holes 15 as suction portions that suck dirt that has been removed from an inner wall inside a body cavity by contact of the balloon 50.

More specifically, as shown in FIG. 2 and FIG. 4, because the aforementioned second tubular portion 6b has a smaller diameter than those of the first tubular portion 6a and the third tubular portion 6c, a peripheral-shaped groove portion 6m as a guiding concave portion is formed at a position adjacent to the balloon 50 that is further on the distal end side in the insertion direction S than the balloon 50 on the outer peripheral surface along the insertion direction S of the distal end portion 6. The plurality of suction holes 15 open in the groove portion 6m, i.e. the second tubular portion 6b.

In this connection, the groove portion 6m is configured so as to facilitate the accumulation of dirt that falls down from the inner wall at a position further on the distal end side in the insertion direction S than the balloon 50 by contact of the balloon 50 with the inner wall inside the body cavity when the insertion portion 4 is advanced in a state in which the balloon 50 is expanded. More specifically, dirt that falls down from the inner wall by contact between the balloon 50 and the inner wall is guided into the groove portion 6m.

The plurality of suction holes 15 are formed at uniform intervals all around the outer periphery of the second tubular portion 6b. More specifically, as shown in FIG. 3, the plurality of suction holes 15 are provided in the second tubular portion 6b such that at least one suction hole 15 is disposed with respect to each of four quadrants Q1 to Q4 as viewed in a planar manner in a state in which a center P in the insertion direction S of the insertion portion 4 is split by two orthogonal lines L1 and L2.

Further, as shown in FIG. 4, the plurality of suction holes 15 communicate to an annular space 45 as shown in FIG. 3 that is covered with the outer periphery of a mouthpiece 6ak of the first tubular portion 6a, the inner circumference of the second tubular portion 6b, and the distal end surface 6cs of the third tubular portion 6c.

As shown in FIG. 3 and FIG. 4, an opening 15h on the distal end side in the insertion direction S of the suction duct 15a opens in the distal end surface 6cs of the third tubular portion 6c so as to face the annular space 45. Thus, the annular space 45 communicates with the suction duct 15a. More specifically, the plurality of suction holes 15 communicate with the single suction duct 15a.

By driving of the suction pump 34, the plurality of suction holes 15 suck in dirt that accumulates in the groove portion 6m by suction power via the communicating annular space 45, suction duct 15a, mouthpiece 19a, suction tube 71, and tube 77.

In this connection, the reason the plurality of suction holes 15 are formed in the groove portion 6m is that, by making it difficult for the plurality of suction holes 15 to come in contact with an inner wall within the body cavity, a situation is prevented in which, along with suction, the plurality of suction holes 15 contact an inner wall inside the body cavity and suck in the inner wall.

Further, the reason the plurality of suction holes 15 are formed at uniform intervals all around the outer periphery of the second tubular portion 6b is to prevent a situation in which all of the plurality of suction holes 15 are blocked at the same time by the inner wall inside the body cavity or dirt.

Furthermore, the reason the plurality of suction holes 15 communicate with the single suction duct 15a using the annular space 45 is that, when performing suction, since suction is continued from other suction holes even if one suction hole 15 contacts an inner wall inside the body cavity, it is possible to prevent a problem such as the advance of the insertion portion 4 being obstructed due to the inner wall that is contacted being attached by suction to a suction hole.

It is necessary that the plurality of suction holes 15 be formed in a known downward direction at least the bend of the bending portion 7 at the outer periphery of the second tubular portion 6b. This is because, since there are many cases in which the downward direction at the bend in the bending portion 7 corresponds to the direction of the force of gravity that works on the distal end portion 6, particularly when the insertion portion 4 is inserted into the intestine of a subject in the supine position, when the suction holes 15 are formed in the downward direction in the outer periphery of the second tubular portion 6b, it is easy to suck in dirt that falls from the intestinal wall in the downward direction of the gravity inside the intestine.

Furthermore, by utilizing the fact that there are many cases in which the downward direction at the bend in the bending portion 7 matches the direction of the force of gravity that works on the distal end portion 6, when a suction hole 15 is also formed in the upward direction at the bend of the bending portion 7 at the outer periphery of the second tubular portion 6b, it becomes easier to establish a distance between the suction hole provided in the upward direction and the intestinal wall. Consequently, there is also an effect that dirt that has accumulated in the groove portion 6m can be securely sucked in without a suction hole on the downward side sucking in the inner wall of the intestine even if the suction hole approaches the intestinal wall to touch the intestinal wall.

Since the positional relationship between the balloon 50 and the plurality of suction holes 15 is not changed by bending of the bending portion 7 or the like because the balloon 50 and the plurality of suction holes 15 are provided at the rigid distal end portion 6, even if the balloon 50 is expanded, the plurality of suction holes 15 are prevented from sucking in the inner wall inside the body cavity.

As shown in FIG. 3, the image pickup cable 80, the light guide 81, the air supply/water supply conduit 82, and the second suction duct 83 and the like are passed through inside the second tubular portion 6b.

Further, as shown in FIG. 3 and FIG. 4, an opening (hereunder, referred to as “water supply conduit opening portion”) 25 on the distal end side in the insertion direction S of the water supply conduit 25a as a liquid supply portion opens at the distal end surface 6cs of the third tubular portion 6c so as to face the groove portion 6m and the plurality of suction holes 15.

By selectively supplying a liquid that is supplied via the tube 73, the mouthpiece 19c, and the water supply conduit 25a when the water supply pump unit 40 is driven to dirt that accumulates in the groove portion 6m, the water supply conduit opening portion 25 softens high viscosity dirt which is difficult for the plurality of suction holes 15 to suck in which accumulates in the groove portion 6m. The water supply conduit opening portion 25 thereby facilitates sucking in of the dirt from the plurality of suction holes 15.

As shown in FIG. 2 to FIG. 4, the air supply opening portion 14 that communicates with the air supply conduit 14a is formed on the outer peripheral surface of the first tubular portion 6a on the distal end portion 6. In this connection, a through hole that penetrates in the diametrical direction through which the air supply conduit 14a passes through is formed in the first tubular portion 6a.

After the commencement of suction of dirt from the plurality of suction holes 15 using the suction pump 34, automatically, the air supply opening portion 14 supplies into the body cavity a gas that is supplied through the air supply tube 72, the mouthpiece 19b, and the air supply conduit 14a by the air supply pump 35 that is driven by means of driving control of the control circuit 32 in an amount that is equal to the amount of gas sucked in from the plurality of suction holes 15.

Thus, the lumen inside the body cavity is prevented from contracting along with suction from the plurality of suction holes 15. As a result, at the time of suction from the plurality of suction holes 15, in addition to enhancing observability from the observation window 11, the insertability of the insertion portion 4 is also improved.

Next, the action of the endoscope of the present embodiment that is configured in this manner is described in brief.

Firstly, after inserting the insertion portion 4 into a body cavity, for example, the large intestine, to facilitate advancement of the insertion portion 4 to a deeper portion of the large intestine, the operator inputs an operation signal to the balloon controller 60 by operating the foot switch 61.

Thereafter, gas is supplied to the balloon 50 from the balloon controller 60 through the tube 74, the mouthpiece 19d, and the balloon duct 50a, to thereby expand the balloon 50. In this connection, expansion of the balloon 50 is performed until the balloon 50 contacts the intestinal wall.

In a state in which the balloon 50 contacts against the intestinal wall, the operator advances the insertion portion 4 to a deeper portion of the intestine. At this time, advance of the insertion portion 4 may be performed by manipulation by the operator or by an automatic insertion device. Performing insertion in a state in which the balloon is expanded ensures that the intestinal tract is constantly widened by the balloon, and thus the state of the lumen can be easily recognized.

Further, by contact of the balloon 50 to the intestinal wall, dirt that adheres to the intestinal wall starts to fall down at a position that is further on the distal end side in the insertion direction S than the balloon 50. As a result, the dirt that falls down adheres to the observation window 11 and the illumination window 13 and it is difficult to carry out illumination and observation inside the intestine. In this connection, the dirt that falls down also starts to accumulate in the groove portion 6m of the distal end portion 6.

Next, the operator operates the air supply/water supply operation button 16 of the operation portion 3 to supply a gas or a liquid from the air supply/water supply pump 23 through the tube 75 or tube 76, the mouthpiece 19e or mouthpiece 19f and the air supply/water supply conduit 82. The supplied gas or liquid is delivered onto the observation window 11 from the nozzle 12. As a result, dirt that adheres to the observation window 11 or the illumination window 13 is removed to secure the field of view.

Subsequently, the operator operates the suction operation button 17 of the operation portion 3 to drive the suction pump 24 to suck in dirt that is removed from the observation window 11 or the illumination window 13 from the second suction portion of the distal end surface 6s through the second suction duct 83, the mouthpiece 19g, and the tube 78.

In this connection, when usage is continued in this state, it is necessary to continuously perform delivery of gas or liquid from the nozzle 12 and suction from the second suction portion. Further, in some cases, dirt accumulates on the distal end surface 6s side while usage of the endoscope 2 is continuing and it may not be possible to remove dirt on the observation window 11 or the illumination window 13 by delivering gas or liquid from the nozzle 12.

Therefore, the operator operates the foot switch 31 to input an operation signal to the suction pump unit 30. Thereafter, by driving the suction pump 34, suction of dirt that has been accumulated in the groove portion 6m commences from the plurality of suction holes 15 via the tube 77, the suction tube 71, the mouthpiece 19a, and the suction duct 15a. In this connection, the performance of suction by the suction pump 34 is not limited to being triggered by input of an operation signal using the foot switch 31 and, for example, may be automatically performed on start-up of the image pickup apparatus.

The dirt that is sucked in is gathered inside the suction bottle 70 through the suction duct 15a, the mouthpiece 19a, and the suction tube 71. Further, when the viscosity of dirt is high and it is difficult to suck in the dirt from the suction holes 15 by operating the foot switch 41, the operator can supply liquid to the dirt that has been accumulated in the groove portion 6m from the water supply conduit opening portion 25 via the tube 73, the mouthpiece 19c, and the water supply conduit 25a from the water supply pump unit 40. As a result, the high-viscosity dirt is softened enough to be sucked from the plurality of suction holes 15.

After starting suction from the plurality of suction holes 15, dirt that falls from the intestinal walls is sucked in whenever necessary from the plurality of suction holes 15. It is therefore dirt accumulates less at a position that is further on the distal end side in the insertion direction S than the balloon 50 of the endoscope 2, and consequently dirt adheres less the observation window 11 or the illumination window 13.

Since the plurality of suction holes 15 are formed across the outer periphery of the second tubular portion 6b, even if one suction hole 15 is blocked, and since suction is possible from the other suction holes 15, a case does not arise in which suction can not be performed.

Since the plurality of suction holes 15 are formed on the outer periphery of the second tubular portion 6b, i.e. the groove portion 6m, and it is thus difficult for them to directly contact the intestinal wall, a situation in which the suction holes 15 suck in the intestinal wall during a suction operation is prevented.

Further, since the pressure sensor 36 is provided at a halfway position of the tube 77, in a case in which the pressure sensor 36 detects that an abnormality has arisen in the suction from the plurality of suction holes 15 by detecting a change in suction pressure, the control circuit 32 can stop the suction pump 34. More specifically, by using the pressure sensor 36, the suction pump 34 can continuously perform suction at a constant suction pressure.

Further, when suction using the suction pump 34 is performed, by driving control of the control circuit 32, automatically, supply of air to inside the intestine from the air supply opening portion 14 is started via the air supply tube 72, the mouthpiece 19b, and the air supply conduit 14a from the air supply pump 35.

In this connection, at this time, by pressure detection by the pressure sensor 37, air is supplied from the air supply pump 35 at the pressure at which suction is performed by the suction pump 34. That is, the same amount of air that is sucked in by the suction pump 34 is supplied to the inside of the intestine from the air supply pump 35. As a result, even when suction is performed with the suction pump 34 through the plurality of suction holes 15, since the inside of the intestine is not flattened, the internal diameter of the intestine can be maintained in a state that is optimal for observation and advancement of the insertion portion 4.

Thus, according to the present embodiment a configuration is described in which the plurality of suction holes 15 that suck in dirt that falls from the inner wall by contact of the expanded balloon 50 and the inner wall inside the body cavity are provided at a position adjacent to the balloon 50 that is further on the distal end side in the insertion direction S than the balloon 50, and further on the proximal end side in the insertion direction S than the observation window 11 or the illumination window 13 at the distal end portion 6 of the insertion portion 4 of the endoscope 2.

It is therefore possible to securely and suck in as needed from the plurality of suction holes 15 dirt that falls down from the inner wall within the body cavity that hinders observation or illumination by adhering to the surface of the observation window 11 or the illumination window 13, that is difficult to suck in with the second suction portion of the second suction duct 83 that opens at the distal end surface 6s of the distal end portion 6. Consequently, even when the balloon 50 is expanded and the insertion portion 4 is caused to advance while contacting the balloon 50 to the inner wall, it is possible to securely prevent dirt from adhering to the surface of the observation window 11 or the illumination window 13 and deteriorating observability.

Thus, the endoscope 2 can be provided that has a configuration that, even in a case in which the insertion portion is advanced inside a body cavity in a state in which the balloon 50 contacts the inner wall within the body cavity, can securely prevent dirt that falls from the inner wall within the body cavity due to contact of the balloon 50 from adhering to the observation window 11 and the illumination window 13.

Hereunder, a modification example is described.

Although according to the present embodiment the plurality of suction holes 15 are given as an example of the suction portion that sucks in dirt that falls from an inner wall due to contact of the balloon 50 and the inner wall, naturally the suction portion is not limited to suction holes. More specifically, the suction portion may be, for example, a suction groove that passes through the annular space 45 in a periphery on the outer periphery of the second tubular portion 6b.

Further, although according to the present embodiment the suction pump 34 for sucking from the plurality of suction holes 15 and the suction pump 24 for sucking from the second suction portion at the distal end surface 6s of the distal end portion 6 are separately provided, the invention is not limited thereto, and a configuration may be adopted in which suction is performed selectively or simultaneously from the plurality of suction holes 15 and the second suction portion by using a valve or the like with the same pump.

Further, although water is supplied from the water supply conduit opening portion 25 by the water supply pump unit 40, the present invention is not limited thereto, and a configuration may be adopted in which water is supplied from the water supply conduit opening portion 25 using the air supply/water supply pump 23.

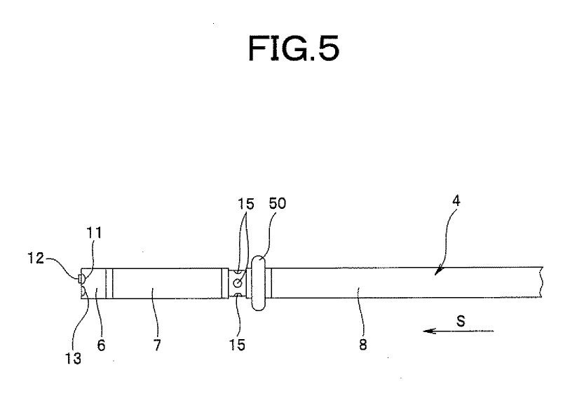

Hereunder, another modification example is described using FIG. 5. FIG. 5 is a diagram showing a modification example in which a balloon and a plurality of suction holes are provided on the proximal end side of the bending portion of the insertion portion of the endoscope, as well as the distal end side in the insertion direction of the insertion portion.

According to the present embodiment, a configuration was described in which the plurality of suction holes 15 are provided across the entire outer periphery of the second tubular portion 6b of the distal end portion 6. However, the present invention is not limited thereto, and as shown in FIG. 5, in a case where the balloon 50 is provided at the proximal end of the bending portion 7, the plurality of suction holes 15 may also be provided at the proximal end of the bending portion 7, as long as the plurality of suction holes 15 are at a position adjacent to the balloon 50 that is further on the distal end side in the insertion direction S than the balloon 50 and further on the proximal end side in the insertion direction S than the observation window 11 or the illumination window 13.

In this case, the balloon 50 and the plurality of suction holes 15 may be formed at an outer periphery position of a known mouthpiece that is connected with the distal end side of the flexible tubular portion 8 on the proximal end side in the insertion direction S of the bending portion 7.

Further, disposition of the balloon 50 and the plurality of suction holes 15 is not limited to the bending portion 7, and a configuration may also be adopted in which the balloon 50 and the plurality of suction holes 15 are provided on the flexible tubular portion 8 that is further on the proximal end side in the insertion direction S than the bending portion 7.

Thus, by providing the balloon 50 and the plurality of suction holes 15 on the proximal end side of the bending portion 7 or between the bending portion 7 and the flexible tubular portion 8, the proximal end side of the bending portion 7 is fixed to the inner wall by contacting the expanded balloon 50 to the inner wall inside the body cavity. Since it is therefore easier to orient the distal end portion 6 in a predetermined direction by a bending operation of the bending portion 7, the insertability of the insertion portion 4 is improved.

Further, even if the insertion portion 4 is advanced when the balloon 50 is in an expanded state, dirt that falls further on the distal end side in the insertion direction S than the balloon 50 from the inner wall along with contact of the balloon 50 can be securely sucked in by the plurality of suction holes 15. The other effects are the same as in the above described embodiment.

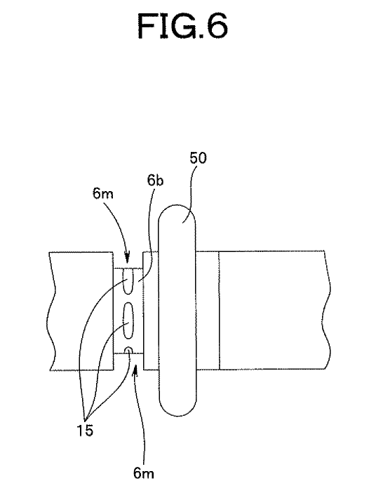

Hereunder, another modification example is described using FIG. 6. FIG. 6 is a diagram showing a modification example in which the plurality of suction holes shown in FIG. 2 are formed as long elliptical holes in the circumferential direction of the distal end portion.

As shown in FIG. 6, the shape of the plurality of suction holes 15 is not limited to the circular shape shown in FIG. 2 and each suction hole 15 may be formed in a long elliptical shape in the circumferential direction of the second tubular portion 6b of the distal end portion 6.

Thus, since the opening area of each suction hole is greater than in the case of a circular hole when each of the plurality of suction holes 15 is formed in an elliptical shape, it is possible to more securely prevent the plurality of suction holes 15 from sucking in the inner wall within the duct when performing suction in comparison to the present embodiment.

In this connection, the shape of the plurality of suction holes 15 is naturally not limited to an elliptical shape as long as the opening area is larger than for a circular shape. The other effects are the same as in the above described embodiment.

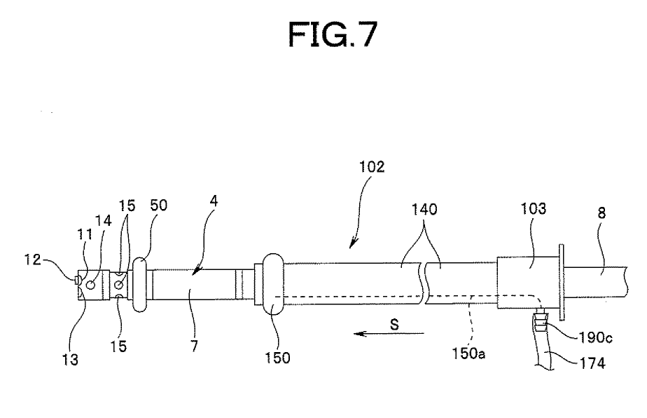

Hereunder, another modification example is described using FIG. 7. FIG. 7 is a diagram showing a modification example in which the endoscope according to the present embodiment is applied to a double-balloon endoscope.

The configuration of the endoscope 2 according to the present embodiment may also be applied to a known double-balloon endoscope 102. More specifically, as shown in FIG. 7, an oversheath 140 is slidably covered over the outer periphery of the insertion portion 4 so that the insertion portion 4 is freely advanceable in the insertion direction S, and a balloon 150 is provided on the outer periphery on the distal end side in the insertion direction S of the oversheath 140.

In this connection, other than the points that the oversheath 140 is covered over the outer periphery of the insertion portion 4, that a mouthpiece 190c for expanding the balloon 150 is provided in the operation portion 103 of the oversheath 140, and that a balloon duct 150a that allows the balloon 150 and the mouthpiece 190c to communicate is provided inside the oversheath 140, the configuration of the double-balloon endoscope 102 is the same as that of the endoscope 2.

More specifically, as shown in FIG. 7, similarly to the present embodiment, in the double-balloon endoscope 102 also the plurality of suction holes 15 are provided at a position that is further on the distal end side in the insertion direction S than the balloon 50 and further on the proximal end side in the insertion direction S than the observation window 11 or the illumination window 13 on the insertion portion 4. Further, the mouthpiece 190c is connected to the balloon controller 60 through a tube 174.

In the double-balloon endoscope 102, when inserting the insertion portion 4 into a body cavity, for example the intestine and advancing the insertion portion 4 therein, first, in addition to inserting the oversheath 140 into the intestine together with the insertion portion 4, the balloon 150 of the oversheath 140 is expanded by supplying air through the tube 174, the mouthpiece 190c, and the balloon duct 150a from the balloon controller 60 to cause the balloon 150 to contact the intestinal wall. At the same time, the balloon 50 is expanded by supplying air from the balloon controller 60.

In this state, when the oversheath 140 is pulled to the proximal end side in the insertion direction S, an intestine that has a curved section changes into a straight shape. As a result, it becomes easier to advance the insertion portion 4 inside the intestine whose shape has changed into a straight shape.

Further, when advancing the insertion portion 4, if insertion is performed in a state in which the expansion of the balloon 50 is made a little loose so that the fixing force with respect to the intestinal wall of the balloon 50 decreases, the lumen enters a widened state to enable insertion in a state in which the field of view is favorable.

In this case also, by performing suction from the plurality of suction holes 15, it is possible to prevent removed dirt that falls from the intestinal wall by contact between the balloon 50 and the intestinal wall from adhering to the observation window 11 or the illumination window 13, similarly to the present embodiment. The other effects are the same as those in the above described embodiment.

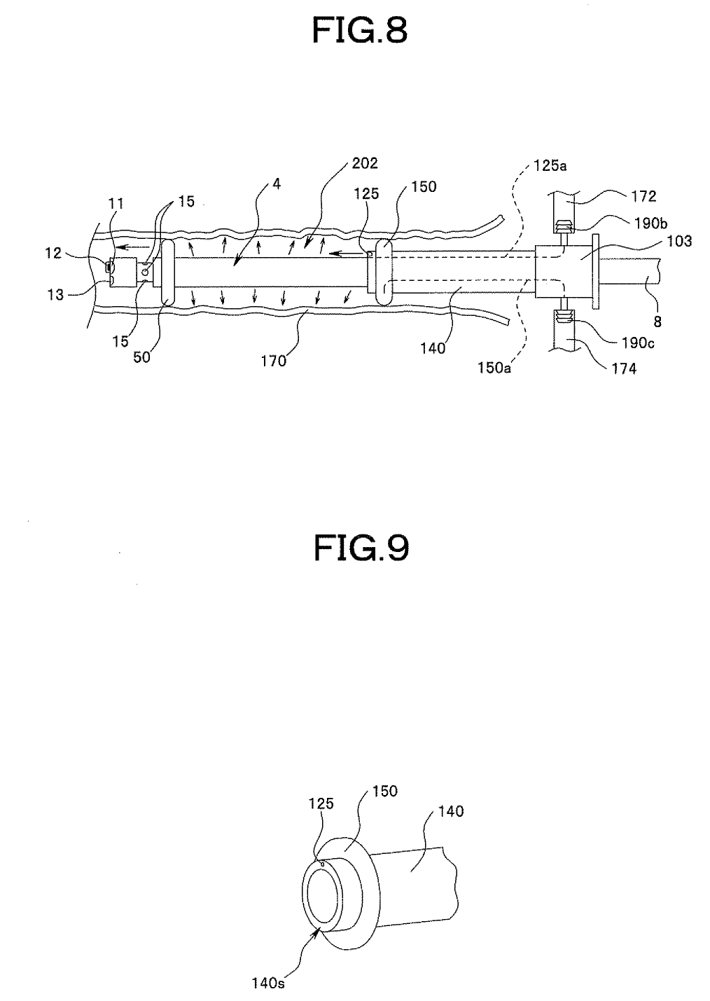

A further modification example is described hereunder using FIG. 8 and FIG. 9. FIG. 8 is a diagram showing a modification example in which the endoscope according to the present embodiment is applied to a self-propelled double-balloon endoscope. FIG. 9 is a perspective view that illustrates an air supply conduit opening portion that is formed in a distal end surface of an oversheath shown in FIG. 8.

The configuration of the endoscope 2 according to the present embodiment may be applied to a known self-propelled double-balloon endoscope 202. More specifically, as shown in FIG. 8, the oversheath 140 is covered over the outer periphery of the insertion portion 4 so that the insertion portion 4 is advanceable in the insertion direction S, and the balloon 150 is provided on the outer periphery of the distal end side in the insertion direction S of the oversheath 140.

Further, as shown in FIG. 9, an opening 125 that is a fluid supply portion of an air supply conduit 125a provided inside the oversheath 140 is formed in a distal end surface 140s of the oversheath 140.

The air supply conduit 125a is connected to the air supply pump 35 by a mouthpiece 190b that is provided in the operation portion 103 of the oversheath 140 and a tube 172 that is connected to the mouthpiece 190b.

More specifically, air that is supplied from the air supply pump 35 is fed into the intestine from the opening 125 through the tube 172, the mouthpiece 190b, and the air supply conduit 125a. The remaining configuration of the self-propelled double-balloon endoscope 202 is the same as that of the endoscope 2.

As shown in FIG. 8, after the insertion portion 4 and the oversheath 140 of the self-propelled double-balloon endoscope 202 are inserted into the body cavity, the balloon 50 and the balloon 150 are expanded to come into contact with an intestinal wall 170. In this state, area between the balloon 50 and the balloon 150 inside the intestine is in a hermetic state.

Subsequently, when the air supply pump 35 is driven, air that is fed from the air supply pump 35 is supplied to the space inside the intestine that is made hermetic by the balloon 50 and the balloon 150 from the opening 125 through the tube 172, the mouthpiece 190b, and the air supply conduit 125a.

Thereafter, when the proximal end side in the insertion direction S of the oversheath 140 is fixed, the insertion portion 4 starts to advance in the insertion direction S by means of the pressure of air that is supplied from the opening 125. As a result, the insertability of the insertion portion 4 is improved.

In this case also, by performing suction from the plurality of suction holes 15, it is possible to prevent dirt that falls from the intestinal wall 170 due to contact between the balloon 50 and the intestinal wall 170 from adhering to the observation window 11 or the illumination window 13, similarly to the present embodiment.

In this connection, the configuration is not limited to one in which a gas such as air is supplied from the opening 125, and a liquid may also be supplied therefrom. The other effects are the same as those in the above described embodiment.

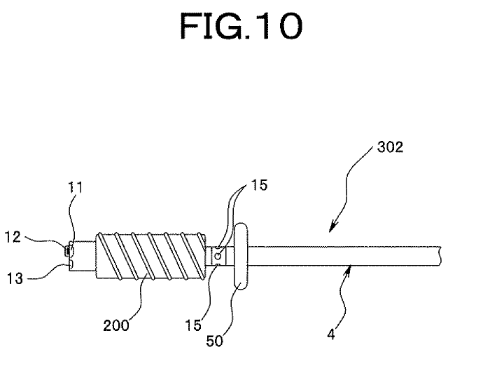

A further modification example is described hereunder using FIG. 10. FIG. 10 is a view showing a modification example in which an endoscope according to the present embodiment is applied to a self-propelled endoscope.

The configuration of the endoscope 2 according to the present embodiment may also be applied to a known self-propelled endoscope 302. More specifically, as shown in FIG. 10, a helical shaped portion 200 as a freely rotatable propulsive force generating portion is provided at a position that is further on the distal end side in the insertion direction S than the plurality of suction holes 15 and further on the proximal end side in the insertion direction S than the observation window 11 or the illumination window 13 on the insertion portion 4.

The helical shaped portion 200 causes the insertion portion 4 to advance inside the intestine by a known screw action by contacting the intestinal wall along with rotation.

Thus, for the self-propelled endoscope 302 that causes the insertion portion to advance along with rotation of the helical shaped portion 200 also, by performing suction from the plurality of suction holes 15 it is possible to prevent dirt that falls from the intestinal wall due to a contact between the balloon 50 and the intestinal wall from adhering to the observation window 11 or the illumination window 13, similarly to the present embodiment.

Further, when dirt that falls from the intestinal wall adheres to a helical portion of the outer periphery of the helical shaped portion 200, the contact force between the helical shaped portion 200 and the intestinal wall decreases and as a result the propulsion force of the helical shaped portion 200 also decreases. However, by performing suction from the plurality of suction holes 15 it is possible to securely prevent dirt that falls from the intestinal wall from adhering to the helical portion of the outer periphery of the helical shaped portion 200. The other effects are the same as in the above described embodiment.

Although an example is described in FIG. 10 in which the self-propelled endoscope 302 that is equipped with the helical shaped portion 200 is used as a self-propelled endoscope, the propulsive force generating portion is not limited to the helical shaped portion 200, and naturally any other kind of propulsive force generating portion may be used.

Further, although according to the present embodiment a direct-viewing type endoscope is given as an example of the endoscope, the present invention is not limited thereto, and a similar effect to the present embodiment can be obtained by applying the present embodiment to a side-view endoscope.

Furthermore, although according to the present embodiment an endoscope for medical treatment is given as an example of the endoscope and a case is described in which the endoscope is inserted into a body cavity, the present invention is not limited thereto, and naturally a similar effect to the present embodiment can be obtained by applying the present embodiment to a case in which an endoscope for industrial use is inserted into a duct.

### Additional Embodiments

As described in detail above, according to the present embodiment of the present invention the configurations described hereunder can be obtained. That is:

(1) An endoscope comprising:

at least one fixing member that is provided on the perimeter of an insertion portion and which protrudes in the diametrical direction; and

at least one suction hole that is provided adjacent to a boundary on a distal end side between the fixing member and the insertion portion.

(2) The endoscope according to additional embodiment 1, wherein

the fixing member is formed with a flexible balloon.

(3) The endoscope according to additional embodiment 2, wherein

the balloon is expandable and contractible.

(4) The endoscope according to additional embodiment 3, wherein

a tube-shaped oversheath is slidingly fitted in an insertion direction on an outer periphery of the insertion portion of the endoscope; and

a second balloon that is capable of expansion and contraction is provided on the outer periphery adjacent to a distal end of the oversheath.

(5) The endoscope according to additional embodiment 4, having

a fluid supply portion that supplies a fluid between the second balloon and the balloon that is provided on the insertion portion.

(6) The endoscope according to additional embodiment 2, wherein

the balloon and the suction hole are provided on a common rigid portion provided in the insertion portion.

(7) The endoscope according to additional embodiment 1, wherein

a bending portion that is actively bendable is provided between the fixing member and a distal end portion of the insertion portion.

(8) The endoscope according to additional embodiment 1, wherein

a propulsive force generating portion that generates a propulsive force by means of a frictional force with respect to an intestinal tract is provided between the fixing member and a distal end portion of the insertion portion.

(9) The endoscope according to additional embodiment 1, wherein

at least one water supply conduit opening portion is provided adjacent to the suction hole.

(10) The endoscope according to additional embodiment 1, wherein

at least one air supply opening portion is provided at a position that is further on a distal end side in an insertion direction than the fixing member.

(11) An endoscope apparatus comprising:

an endoscope according to additional embodiment 10;

an air supply amount detection portion that detects an air supply amount from the air supply opening portion;

a suction amount detection portion that detects a suction amount from the suction hole; and

an air supply amount control portion that, based on information of the suction amount detection portion, automatically adjusts an air supply amount and supplies air from the air supply opening portion.

(12) The endoscope according to additional embodiment 1, wherein

a plurality of the suction holes are provided.

(13) The endoscope according to additional embodiment 12, having

at least one suction duct within the insertion portion, wherein the suction duct communicates with at least two of the suction holes.

(14) The endoscope according to additional embodiment 1, wherein

at least one each of the suction holes is disposed with respect to each of four quadrants that are divided by intersecting a center of the insertion portion at right angles.

(15) The endoscope according to additional embodiment 1, wherein

the suction hole is provided in a suction opening installation portion; and

a first outer diameter of the suction opening installation portion is smaller than that of a second outer diameter of an insertion portion that is adjacent at a distal end side in the insertion direction of the suction opening installation portion, and a second outer diameter of the fixing member is larger than that of the second outer diameter.

(16) The endoscope according to additional embodiment 1, wherein

the suction hole has a shape of that is long and narrow in a circumferential direction.

(17) An endoscope apparatus comprising:

an endoscope according to additional embodiment 1;

at least one suction duct with which the suction portion provided in the insertion portion communicates;

a suction apparatus that applies a suction pressure to the suction duct;

a detection portion that detects the suction pressure;

a suction pressure changing portion capable of freely adjusting the suction pressure; and

a suction pressure control portion that controls a suction pressure using the suction pressure changing portion based on information of the detection portion.

Guidance and insertion of a distal end in a lumen direction of an intestine using a fixing member is facilitated by additional embodiment 1 having a configuration as described above. Furthermore, since dirt inside the intestine that accumulates in a fixing member along with insertion can be sucked in and removed as needed, it can not be obstructed by accumulated dirt.

The invention described in the above embodiments is not limited to those embodiments, and various modifications can be made thereto at the execution stage to a degree that does not deviate from the spirit and scope of the invention. Further, inventions of various stages are included in the present embodiments and various inventions can be extracted by suitability combining a plurality of the configurational requirements that are disclosed.

For example, in a case in which the problem set forth as a problem to be solved by the invention can be solved and the effect described as the effect of the invention can be obtained even if several of the configurational requirements are deleted from the entire configurational requirements disclosed in an embodiment, the configuration from which the relevant configurational requirements were deleted can be extracted as an invention.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An endoscope, including:
an insertion portion that is inserted into a duct;
an observation window that is provided at a distal end in an insertion direction of the insertion portion for observing the inside of the duct;
a fixing member provided so as to extend in a radial shape in a diametrical direction of the insertion portion with respect to the insertion portion, and which contacts an inner wall of the duct to fix the insertion portion to the inner wall; and
a suction portion provided at a position that is further on the distal end side in the insertion direction than the fixing member while being further on a proximal end side in the insertion direction than the observation window in the insertion portion, and which sucks in dirt that is removed from the inner wall by contact between the fixing member and the inner wall.

2. The endoscope according to claim 1, wherein
the suction portion is provided at a position adjacent to the fixing member.

3. The endoscope according to claim 1 wherein
the fixing member is a balloon that freely expands and contracts in the diametrical direction along with supply of a fluid and discharging a fluid.

4. The endoscope according to claim 1, wherein
the suction portion is provided on an outer peripheral surface along the insertion direction of the insertion portion.

5. The endoscope according to claim 1, wherein
the fixing member and the suction portion are provided on a distal end rigid portion that is provided at the distal end in the insertion direction of the insertion portion.

6. The endoscope according to claim 1, wherein:
at a position between the fixing member and a distal end rigid portion that is provided at the distal end in the insertion direction of the insertion portion is provided a freely bendable bending portion that orients the distal end rigid portion in a plurality of directions; and
the suction portion is provided in the bending portion.

7. The endoscope according to claim 1, wherein:
a guiding concave portion that guides dirt that is removed by the fixing member is provided at a position adjacent to the distal end side in the insertion direction of the fixing member in the insertion portion; and
the suction portion is provided in the guiding concave portion.

8. The endoscope according to claim 1, wherein
the suction portion is provided in communication with a suction duct that is inserted through the insertion portion and connected to a suction apparatus.

9. The endoscope according to claim 8, wherein:
the suction portion is a suction hole that is opened in the insertion portion; and
a plurality of the suction holes are provided.

10. The endoscope according to claim 9, wherein
the suction duct communicates with at least two of the suction holes.

11. The endoscope according to claim 1, wherein
a liquid supply portion that supplies a liquid to the suction portion is provided at a position adjacent to the distal end side in the insertion direction of the fixing member in the insertion portion.

12. The endoscope according to claim 8, wherein
a second suction portion that is different from the suction portion that sucks in a liquid inside the duct is provided in a distal end surface of the distal end in the insertion direction of the insertion portion, and a second suction duct and that is different to the suction duct communicates with the second suction portion is provided inside the insertion portion.

13. An endoscope, including:
an insertion portion that is inserted into a duct;
an illumination light emitting window that is provided at a distal end in an insertion direction of the insertion portion for illuminating the inside of the duct;
a fixing member provided so as to extend in a radial shape in a diametrical direction of the insertion portion with respect to the insertion portion, and which contacts an inner wall of the duct to fix the insertion portion to the inner wall; and
a suction portion provided at a position that is closer to the distal end side in the insertion direction than the fixing member while being closer to a proximal end side in the insertion direction than the illumination light emitting window in the insertion portion, and which sucks in dirt that is removed from the inner wall by contact between the fixing member and the inner wall.

14. The endoscope according to claim 13, wherein
the suction portion is provided at a position adjacent to the fixing member.

15. The endoscope according to claim 13, wherein
the fixing member is a balloon that freely expands and contracts in the diametrical direction along with feeding a fluid or discharging a fluid.

16. The endoscope according to claim 13, wherein
the suction portion is provided on an outer peripheral surface along the insertion direction of the insertion portion.

17. The endoscope according to claim 13, wherein
the fixing member and the suction portion are provided on a distal end rigid portion that is provided at the distal end in the insertion direction of the insertion portion.

18. The endoscope according to claim 13, wherein:
at a position between the fixing member and a distal end rigid portion that is provided at the distal end in the insertion direction of the insertion portion is provided a freely bendable bending portion that orients the distal end rigid portion in a plurality of directions; and
the suction portion is provided in the bending portion.

19. The endoscope according to claim 13, wherein:
a guiding concave portion that guides dirt that is removed by the fixing member is provided at a position adjacent to the distal end side in the insertion direction of the fixing member in the insertion portion; and
the suction portion is provided in the guiding concave portion.

20. The endoscope according to claim 13, wherein
the suction portion is provided in communication with a suction duct that is inserted through the insertion portion and connected to a suction apparatus.

21. The endoscope according to claim 20, wherein:
the suction portion is a suction hole that is opened in the insertion portion; and
a plurality of the suction holes are provided.

22. The endoscope according to claim 21, wherein
the suction duct communicates with at least two of the suction holes.

23. The endoscope according to claim 13, wherein
a liquid supply portion that supplies a liquid to the suction portion is provided at a position adjacent to the distal end side in the insertion direction of the fixing member in the insertion portion.

24. The endoscope according to claim 20, wherein
a second suction portion that is different from the suction portion that sucks in a liquid inside the duct is provided in a distal end surface on the distal end in the insertion direction of the insertion portion, and a second suction duct that is different from the suction duct and communicates with the second suction portion is provided inside the insertion portion.
